(19) United States
(12) Patent Application Publication
(10) Pub. No.: US 2017/0071990 A1
(43) Pub. Date: Mar. 16, 2017

(54) COMPOSITIONS CONTAINING BORIC ACID AND A MIXTURE OF LACTOBACILLUS

(71) Applicant: Pizeta Group Srl, Perugia (IT)

(72) Inventors: Francesco DE SETA, Trieste (IT); Lucio LEONARDI, Perugia (IT)

(73) Assignee: Pizeta Group Srl, Perugia (IT)

(21) Appl. No.: 15/311,360

(22) PCT Filed: May 6, 2015
(86) PCT No.: PCT/IB2015/053300

§ 371 Date: Nov. 15, 2016

(30) Foreign Application Priority Data
May 16, 2014 (IT) MI2014A000895

(51) Int. Cl. A61K 35/747; A61K 9/00; A61K 33/22

(52) U.S. Cl. CPC A61K 35/747; A61K 33/22; A61K 9/0034

(57) ABSTRACT

The present invention relates to a new composition, containing boric acid and a mixture of at least two lactobacilli selected from L. rhamnosus, L. crispatus, L. jensenii and L. gasseri. The present invention further relates to the use of the above composition in the treatment of fungal and/or bacterial vulvovaginitis.

### STATE OF THE ART

The vaginal bacterial flora is characterized by a set of micro-organisms in dynamic equilibrium with the environment, whose composition undergoes frequent and significant changes in the course of a woman's life, mostly reflecting the physiological changes in hormonal balance, particularly the estrogen circulating level, and other factors such as sexual activity, pregnancy, hygienic-sanitary conditions, systemic diseases, pharmacological treatments (with antibiotics or immunosuppressants), radiation therapy and trauma.

In recent years, thanks to the HMP project (The Human Microbiome Project), the micro-organisms composition in the vaginal microenvironment has been fully described. A first interesting fact emerging from these studies is that the composition of the vaginal microbial flora is extremely variable in the population, and it is significantly influenced not only by environmental factors, but also by the host organism itself, in particular by its genotype, sex, age and state of immune maturation. However, there is a core of at least 57 bacterial species that can be considered common to all individuals.

In most cases, and especially in the Caucasian race, the typical vaginal microflora during fertile age is dominated by genus Lactobacillus (95%), facultative anaerobic or microaerophilic gram-positive bacteria, characterized in nature by at least 60 species.

Lactobacilli constitute the majority of the group of lactic acid bacteria, so called because almost all of their members convert lactose and other sugars into lactic acid by means of lactic fermentation. Lactobacilli produce lactic acid through sugars fermentation, reducing the pH of the environment in which they grow, acetic acid, ethanol, carbon dioxide, and other secondary compounds. In particular, the acidification of the vaginal environment (pH of about 5) inhibits the growth of certain pathogenic micro-organisms (e.g. Candida albicans).

In humans, lactobacilli are present as symbiotic micro-organisms both in the vagina and the gastrointestinal tract, and represent a small portion of the human microbiota.

The prevalent lactobacilli species present in vaginal microflora are represented by: L. iners, L crispatus, L. gasseri, L. jenesenii; while in lower concentration can be found: L. acidophilus, L. fermentum, L plantarum, L. brevis, L. casei, L. vaginalis, L. delbrueckii, L. salivarius, L. reuteri and L. rhamnosus.

Together with the local immune system, the main lactobacilli play a key role in host defense, both through the formation of a “biofilm” that prevents the adhesion of pathogens and competition for metabolites.

By “biofilm” it is meant a complex aggregation of micro-organisms characterized by the secretion of a protective and adhesive matrix.

In particular, lactic acid is the most important limiting factor of vaginal colonization by exogenous micro-organisms, and the growth of potentially pathogenic commensals, and in inhibiting the activity of bacterial virulence factors, such as sialidase and mucinase, enzymes that play a crucial role in eluding the local immune response.

Lactic acid bacteria, mostly represented by lactobacilli are together with the bifid bacteria the most common types of micro-organisms used as probiotics, live micro-organisms which administered in adequate amounts confer a health benefit on the host.

The probiotic activity of lactobacilli differs, not only by species, but is also strain-specific.

The main properties that lactobacilli must have to be suitable for use as probiotics are:
an appropriate amount to ensure a sufficient degree of vaginal colonization;
ability to adhere to the urogenital epithelium forming a biofilm, which covers the walls of the local mucosa, able to favor both consolidation of lactobacilli colonization and protection of the mucosa from the aggression of micro-organisms responsible for infections;
an appropriate antibacterial activity;
survival at a pH<4.5, and at a temperature different from the physiological one, or otherwise not optimal for the lactobacilli;
ability to resist the antibiotics used in the treatment of urogenital infections.

The criteria described above have enabled the development of probiotics useful for maintaining the health of the vagina, and specifically highlighted the action of the different lactobacilli strains. For example, L. rhamnosus is able to inhibit the growth of Gram negative pathogens and Candida, while lactobacilli L. crispatus and L. jensenii, mainly represented in physiological conditions, are implicated in the pathogenesis of bacterial vaginosis since, through the production of biosurfactants, they oppose the adhesion of Gram-positive and anaerobic micro-organisms.

It is known that conditions of dysbiosis, which involve an alteration of the bacterial flora, or pathological conditions, such as bacterial vaginosis and/or fungal vaginitis, determine a disruption of the vaginal microbial populations and may be related to an increased risk of developing sexually transmitted infection diseases, both of viral and bacterial origin.

It is indeed observed that, when there is a disruption of the vaginal ecosystem, with a consequent reduction of the normal lactobacilli flora, an increase of bacterial species represented by Gardnerella vaginalis, Atopobium vaginae and Gram-negative anaerobic bacteria (for example, Prevotella, Peptostreptococcus, Mycoplasma hominis, Ureaplasma urealyticum, Mobiluncus and Bacteroides) is observed.

Moreover, women with bacterial vaginosis show a dramatic decrease in the concentration of L. crispatus, and the acquisition of a complex microbial ecosystem consisting mainly of anaerobic micro-organisms, such as Gardnerella and Prevotella producing a particular biofilm responsible for recurrent bacterial vaginosis.

It was also demonstrated that the use of L. rhamnosus GR-1 allows to replenish the lactobacillus environment disrupted by bacterial infections, with good results in reducing the recurrence of bacterial vaginosis when administered as an adjunct to metronidazole. L. rhamnosus GR-1 is, in fact, able to penetrate within the biofilm built by Gardnerella vaginalis and Atopobium vaginae and cause their destruction.

Peroxide producing lactobacilli, such as L. crispatus, L. jensenii, are also strongly reduced by bacterial vaginosis, while L. crispatus possess and excellent adhesion to the vaginal cells, enabling it to permanently remain in the vaginal microbiome and prevent the binding of pathogens to the cell membrane. It was demonstrated that bacterial vaginosis incidence is reduced by four times in patients colonized in the rectum and the vagina by peroxide producing lactobacilli, particularly by L. crispatus and L. jensenii, which are normally present at both sites in the 37% of women.

However, in order to rebalance the vaginal microenvironment, experimental evidence (May A. D and Hillier S. The Journal of Infectious Diseases 2005; 192:394-8) suggests using probiotics based on lactobacilli that are usually predominant in the healthy vagina, due to their increased capacity to permanently colonize the vagina.

With regards to fungal infections of the urogenital apparatus, the prevalence of Vulvovaginal Candidiasis (VVC) ranges between 20 and 30%, depending on the population studied, with 13 million cases per year in the United States alone. The most affected age is the fertile age.

While various species of Candida exist from a taxonomical point of view, only ten of them are pathogenic to humans. It is estimated that, in 70% cases, VVC is supported by C. albicans, in 20% by C. glabrata, in 10% by C. krusei and/or C. tropicalis, while the other species (C. pseudotropicalis, C. lusitaniae, C. rugoso, C. parapsilopsis) are isolated in less than 1% of cases.

From a clinical point of view, the various species mentioned are similar, with the difference being that non albicans variants more frequently cause relapsing forms, invasive candidiasis and candidemia.

For therapeutic purposes, the guidelines divide VVC into two broad categories, characterized by a different drug response: uncomplicated forms and complicated forms.

Uncomplicated forms of VVC represent 90% of all VVC and, generally, they are responsive to all short term therapeutic regimens (1-7 days), both local and systemic, with a rapid resolution of symptoms (48-72 hours), and culture negativization in 80-90% of patients who completed the therapy.

Complicated forms of VVC have more severe symptoms, and include recurrent forms and the forms supported by non albicans Candida species. Typically, they affect patients with well-known risk factors, such as unbalanced diabetes mellitus, immunosuppression, broad-spectrum antibiotic therapy protracted in time. Literature data show that some specific strains of lactobacilli, such as L. rhamnosus GR-1 and L. reuteri RC-14, are effective in suppressing the growth of Candida albicans and killing the fungus at low pH (4.5) conditions, and in an environment rich in lactic acid (Köhler G A, Assefa S, Reid G. Infect Dis Obstet Gynecol. 2012; pp. 636474), thanks to lactic acid production that allow to maintain a low pH able to inhibit C. albicans growth. In fact, undissociated lactic acid crosses the plasmatic barrier through diffusion, and only subsequently dissociates into ions, determining acidification of the cytosol that interferes with the fungus metabolic pathways.

The production of hydrogen peroxide by lactobacilli seems to be an additional, but not crucial, mechanism, since there is no difference in the in vitro inhibition of the fungus growth between L. reuteri (peroxide producer) and L. rhamnosus (non-peroxide producer).

A number of in vitro studies have demonstrated, in fact, how lactobacilli such as L. delbrueckii, L. plantarum, L. acidophilus, L. gasseri are able to inhibit adhesion and/or growth of Candida albicans through production of biosurfactants or similar bactericide substances.

In addition, it has been observed that probiotics are able to increase the colonization of peroxide producing lactobacilli: L. crispatus, L. jensenii and L. vaginalis, reducing the incidence of vulvovaginal candidiasis and bacterial vaginosis.

In recent years, it has been seen an increasing spread of so-called nutraceuticals, probiotics and functional food which, in addition to nutritional properties, have scientifically demonstrated the ability to positively influence one or more body functions, while helping to preserve or improve health and well-being, and reducing the risk of disease outbreaks in the population.

In particular, there has recently been a growing increase in studies directed to test and measure the effectiveness of probiotics in the treatment and/or prevention of the lower female urogenital tract infections, especially against Candida and bacterial vaginosis infections, which are the most common diseases affecting women in the fertile age.

The beneficial effects of probiotics on health are mainly related to their ability to modulate and stabilize the composition of the vaginal flora, as well as to help the host fight any pathogens present in the urogenital tract.

In the treatment of such infections, and especially in complicated and uncomplicated forms of Candida albicans, it was also noted that boric acid, or orthoboric acid (H₃BO₃), is a therapeutic presidium of proven effectiveness. Boric acid, a weak acid often used as antiseptic, insecticide or disinfectant, is rapidly and completely absorbed in the body after oral administration, it is widely distributed in body fluids through passive diffusion, and accumulates in bones, brain, liver, and kidneys. In particular, studies on vaginal absorption after administration of vaginal suppositories containing 600 mg/ovule of boric acid, have shown irrelevant circulation levels of boric acid (under 1 μg/ml) throughout the duration of the treatment (Seigel E et al., Boric acid toxicity. Ped Clin North Am 1986; 33: 363-67 and Jansen J A et al., Boric acid single dose pharmacokinetics after intravenous administration to man. Arch Toxicol 1984; 55: 64-67).

Boric acid usually has a very low acute toxicity in both animals and humans, and no mutagenic or teratogenic properties were observed in in vitro and in vivo studies (Di Renzo F et al., Boric acid inhibits histone embryonic histone deacetylases: A suggested mechanism to explain boric acid-related teratogenicity. Toxicol. Appl. Pharmacol. 2007; 220: 178-85), in particular, concentrations of boric acid up to 5% are considered safe.

Fungicidal properties of boric acid have been long time exploited in the gynecological field for the treatment of mycotic vulvovaginitis.

Although its exact mechanism of action has not been fully clarified yet, boric acid has a proven fungistatic and moderately bacteriostatic action.

It was initially assumed that, in case of mycotic vulvovaginitis, the action of boric acid could be explained by the acidification of the vaginal environment. In vitro tests have, however, demonstrated that the pH at which the substance completely inhibits the growth of Candida albicans is usually between 5-5.9, values very similar to the physiological vaginal pH, thus demonstrating that the mechanism of acidification alone is not sufficient to demonstrate the fungistatic properties of boric acid.

Differently from what reported in the past literature, it was demonstrated that the fungistatic action of boric acid is not to be connected to an action mediated by the pH, but to a series of metabolic actions.

It was observed that low concentrations of boric acid in non-therapeutic doses, and for amounts of time limited to the first 24 hours, are able to interfere with different factors that contribute to determine the mechanisms of virulence of Candida, and determine not only a decrease of cellular ergosterol, resulting in a fungistatic action, but above all a reduction of the fungus germination processes, and a moderate inhibition of the micro-organisms growth, not only in planktonic but also in biofilm form.

The foregoing opens, as a result, new perspectives for boric acid which, when used al low concentrations and for limited period of times, and in association with other therapeutic (antifungal/antibacterial) or rebalancing (lactobacilli) agents may act as an adjuvant/rebalancing agent as it allows to facilitate their mechanism of action.

In particular, the need for alternative therapeutic approaches arises from the frequent finding of relapses in clinical practice, resulting in significant psychological and physical discomfort for the patient, as they significantly disrupt the quality of life with high healthcare costs.

The failure of the antibiotic/antifungal therapy commonly used to treat the female urogenital tract infections is linked with two main factors, such as the development of resistance to chemotherapy by pathogens and the negative impact of antibiotics on vaginal microflora, that complicate regeneration of lactobacilli, which are the main line of defense of the vaginal ecosystem in women of fertile age. In fact, an altered bacterial flora facilitates both the colonization by exogenous micro-organisms and the virtualization of potentially pathogenic commensal bacteria, that are usually present at low concentrations.

In order to prevent and counteract the onset of these infections, there is therefore a clear need for a new and improved therapeutic approach for the treatment and prevention of mycotic and bacterial vulvovaginitis, which involves restoring the vaginal ecosystem and that has a greater effectiveness and fewer side effects than the treatments used until now.

### DESCRIPTION

It was surprisingly found that a composition comprising boric acid and a specific mixture of lactobacilli may be advantageous not only for the patients suffering from fungal vaginitis or bacterial vaginosis, but also in cases of infection already treated or recurrent.

The surprising effect is due to the possibility to act against the bacteria responsible for bacterial vaginosis and against the proliferation of Candida Albicans, through a single composition consisting of boric acid and lactobacilli, without the presence boric acid being an obstacle or inactivating lactobacilli. This combination, never described before, sets the basis for rebalancing the vaginal homeostasis and, therefore, a “healthy” vaginal state.

From a therapeutic point of view, it is important to emphasize that boric acid has the ability to improve the action of the lactobacilli mixture, by reducing the fungus germination processes and inhibiting the formation of biofilm, thus promoting the effectiveness of the lactobacilli which constitute the “good” biofilm.

A further action of boric acid is related to the pump mechanisms of the fungus that favor the adhesion of the active substance to the cell wall of the pathogen. The experimental data obtained by the present inventors have demonstrated that boric acid has a good activity against Candida albicans, but has a poor activity against Gardnerella vaginalis; while the lactobacilli tested show a comparable inhibition activity against both Candida albicans and Gardnerella vaginalis. Taking, therefore, into consideration all the alteration profiles of the vaginal bacterial flora with either aerobic vaginitis or bacterial vaginosis, the composition of the present invention results in a synergistic effect of the boric acid, when used in association with a mixture of lactobacilli, on the vaginal homeostasis, and specifically aimed at an inhibition activity against bacterial populations, such as Gardnerella vaginalis, E. coli, streptococci and staphylococci, and fungal populations, such as Candida albicans.

In addition, the mixture of lactobacilli, through biofilm production, release of lactic acid, the action of promoting lactobacilli proliferation, the constant recovery of the physiological vaginal pH and the cervical-vaginal immunomodulation action, result in a strengthening of the vaginal defenses, as well as an optimization of the local defense, aimed at the prevention of recurrence of bacterial dismicrobism and infections, both anaerobic and aerobic.

A composition comprising boric acid with the simultaneous presence of a mixture of at least two lactobacilli selected from L. ramnosus, L. crispatus, L. jensenii and L. gasseri, has therefore the following advantages:

1) restore normal homeostasis in terms of vaginal pH, local immunity and selective bacterial inhibition (aerobic and anaerobic);
2) allow the creation of a selective lactobacilli biofilm which not only has the function of inhibiting the growth of pathogenic commensal bacteria, but also of interfering with the adhesion processes of such micro-organisms at the vaginal mucosal level;
3) assist the action of any antibacterial or antifungal agents used in the course of vulvovaginal infections;
4) prevent relapses or recurrences of infection therefore avoiding indiscriminate and extended use of antibiotics and/or antifungals;
5) assist the vaginal mucosa healing process by promoting re-epithelialization of the tissues.

An object of the present invention is, therefore, a composition comprising boric acid and a mixture of at least two lactobacilli, selected from L. rhamnosus, L. crispatus, L. jensenii and L. gasseri.

The composition of the present invention preferably contains boric acid and a mixture comprising two, three or four of the aforementioned lactobacilli.

Preferably, the composition of the present invention comprises boric acid, L. rhamnosus and L. gasseri.

More preferably, the composition of the present invention comprises boric acid, L. rhamnosus, L. crispatus and L. gasseri.

Even more preferably, the composition of the present invention comprises boric acid, L. rhamnosus, L. crispatus, L. jensenii and L. gasseri.

According to an embodiment of the present invention, boric acid is present in the composition in an amount by weight comprised between 70% and 10%, preferably between 60% and 15%; while the lactobacilli mixture is present in the composition in an amount by weight comprised between 30% and 10%, preferably between 25% and 15%, about 20%.

The above percentage amounts are amounts by weight based on the total weight of the composition.

When the aforementioned mixture of lactobacilli consists of L. gasseri and L. rhamnosus, the lactobacillus L. gasseri is present in said mixture in an amount by weight comprised between 15% and 5%, preferably between 10% and 2%, based on the total weight of the mixture; while the lactobacillus L. rhamnosus is present in said mixture in an amount by weight comprised between 15% and 5%, preferably between 10% and 2%, based on the total weight of the mixture.

When the aforementioned mixture of lactobacilli consists of L. crispatus, L. jensenii and L. gasseri, the lactobacillus L. crispatus is present in said mixture in an amount by weight comprised between 13% and 3%, preferably between 10% and 2%, based on the total weight of the mixture; the lactobacillus L. jensenii is present in an amount by weight comprised between 13% and 3%, preferably between 10% and 2%, based on the total weight of the mixture; the lactobacillus L. gasseri is present in an amount by weight comprised between 13% and 3%, preferably between 10% and 2%, based on the total weight of the mixture.

When the aforementioned mixture of lactobacilli consists of L. crispatus, L. jensenii, L. gasseri and L. rhamnosus, the lactobacillus L. crispatus is present in said mixture in an amount by weight comprised between 11% and 1%, preferably between 8% and 1%, based on the total weight of the mixture; the lactobacillus L. jensenii is present in an amount by weight comprised between 11% and 1%, preferably between 8% and 1%, based on the total weight of the mixture; the lactobacillus L. gasseri is present in an amount comprised between 11% and 1%, preferably between 8% and 1%, based on the total weight of the mixture; the lactobacillus L. rhamnosus is present in an amount by weight comprised between 11% and 1%, preferably between 8% and 1%, based on the total weight of the mixture.

According to an embodiment of the present invention, when the mixture comprises 2 lactobacilli, such as L. rhamnosus and L. gasseri, the total amount of micro-organisms present in the mixture is comprised between 0.3 and 2.5 billion/dose (defined as total lactobacilli claimed at the commercial expiry date), preferably between 0.5 and 2 billion/dose, more preferably between 1 and 1.5 billion/dose.

According to an embodiment of the present invention, when the mixture comprises 3 lactobacilli, such as L. rhamnosus, L. crispatus and L. gasseri, the total amount of micro-organisms present in the mixture is comprised between 0.5 and 3.5 billion/dose, preferably between 0.8 and 3 billion/dose, more preferably between 1 and 2.5.

According to an embodiment of the present invention, when the mixture comprises 4 lactobacilli, such as L. rhamnosus, L. crispatus, L. gasseri and L. jensenii the total amount of micro-organism present in the mixture is comprised between 0.5 and 3.5 billion/dose, preferably between 0.8 and 3 billion/dose, more preferably between 1 and 2.5.

A further object of the present invention is a pharmaceutical formulation comprising the above composition and at least one physiologically acceptable excipient.

The term “physiologically acceptable excipient”, according to the present invention, means a substance without a pharmacological effect of its own and that does not produce any adverse reactions when administered to mammals, preferably to humans.

The composition of the present invention is preferably formulated in a form suitable for vaginal administration.

According to an embodiment of the present invention, said form suitable for vaginal administration is a vaginal capsule, an ovule, a cannula, a gel or a solution for intracavitary use, preferably a vaginal capsule or an ovule.

Preferably, it is a capsule in which the composition comprising boric acid and the mixture of at least two lactobacilli is present in the form of granules, and it is combined with at least one physiologically acceptable ingredient, preferably selected from inulin, maltodextrin, magnesium stearate and silicon dioxide; the coating of the capsule preferably consists of a cellulose ester, even more preferably it consists of hydroxypropylmethylcellulose (HPMC) and titanium dioxide as a dye.

A further object of the present invention is the use of a composition comprising boric acid and a mixture of at least 2 lactobacilli in the treatment and/or prevention of fungal and/or bacterial vulvovaginitis.

Said fungal vulvovaginitis is preferably a Candida vulvovaginitis. Said bacterial vulvovaginitis is, preferably, a Gardnerella vaginalis, E. coli, streptococci and/or staphylococci vulvovaginitis.

According to the invention, the composition of the present invention may be administrated to humans, intended as an adult subject, preferably a female subject. The composition of the present invention is preferably administrated daily, in one to four doses per day.

According to a preferred embodiment of the present invention, said composition is administered for at least ten days, preferably for at least four weeks.

The following examples are for illustrative purposes and are not intended to limit the scope of the present invention.

### EXAMPLES

1. Formulas with 4 probiotic micro-organisms in association with boric acid (scalar concentrations of probiotics and boric acid):

### Example 1a

| Ingredient | mg/dose | Probiotics Supply |
|---|---|---|
| Boric acid | 300 | |
| Lactobacillus crispatus | 100 | 1 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 1b

| Ingredient | mg/dose | Probiotics Supply |
|---|---|---|
| Boric acid | 250 | |
| Lactobacillus crispatus | 100 | 1 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 71 | |
| Magnesium stearate | 4 | |
| Silicon dioxide | 4 | |
| HPMC and titanium dioxide | 96 | |
| capsule | | |
| TOTAL | 550 | |

### Example 1c

| Ingredient | mg/dose | Probiotics Supply |
|---|---|---|
| Boric acid | 200 | |
| Lactobacillus crispatus | 100 | 1 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 121 | |
| Magnesium stearate | 4 | |
| Silicon dioxide | 4 | |
| HPMC and titanium dioxide | 96 | |
| capsule | | |
| TOTAL | 550 | |

### Example 1d

| Ingredient | mg/dose | Probiotics Supply |
|---|---|---|
| Boric acid | 150 | |
| Lactobacillus crispatus | 100 | 1 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 171 | |
| Magnesium stearate | 4 | |
| Silicon dioxide | 4 | |
| HPMC and titanium dioxide | 96 | |
| capsule | | |
| TOTAL | 550 | |

### Example 1e

| Ingredient | mg/dose | Probiotics Supply |
|---|---|---|
| Boric acid | 100 | |
| Lactobacillus crispatus | 100 | 1 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 171 | |
| Magnesium stearate | 4 | |
| Silicon dioxide | 4 | |
| HPMC and titanium dioxide | 96 | |
| capsule | | |
| TOTAL | 500 | |

### Example 1f

| Ingredient | mg/dose | Probiotics Supply |
|---|---|---|
| Boric acid | 300 | |
| Lactobacillus crispatus | 100 | 1.5 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 1h

| Ingredient | mg/dose | Probiotics Supply |
|---|---|---|
| Boric acid | 300 | |
| Lactobacillus crispatus | 100 | 2 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 1i

| Ingredient | mg/dose | Probiotics Supply |
|---|---|---|
| Boric acid | 300 | |
| Lactobacillus crispatus | 100 | 2.5 Billion/dose (total |
| Lactobacillus rhamnosus | | lactobacilli claimed at |
| Lactobacillus gasseri | | the commercial expiry date) |
| Lactobacillus jensenii | | |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

2. Formulas with 3 probiotic micro-organisms in association with boric acid (scalar concentrations of probiotics and boric acid):

### Example 2a

| Ingredient | mg/dose | Probiotics Supply |
|---|---|---|
| Boric acid | 300 | |
| Lactobacillus crispatus | 100 | 1 Billion/dose (total |
| Lactobacillus rhamnosus | | lactobacilli claimed at |
| Lactobacillus gasseri | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 2b

| Ingredient | mg/dose | Probiotics Supply |
|---|---|---|
| Boric acid | 250 | |
| Lactobacillus crispatus | 100 | 1 Billion/dose (total |
| Lactobacillus rhamnosus | | lactobacilli claimed at |
| Lactobacillus gasseri | | the commercial expiry date) |
| Inulin | 25 | |
| Maltodextrin | 71 | |
| Magnesium stearate | 4 | |
| Silicon dioxide | 4 | |
| HPMC and titanium dioxide | 96 | |
| capsule | | |
| TOTAL | 550 | |

### Example 2c

| Ingredient | mg/dose | Probiotics Supply |
|---|---|---|
| Boric acid | 200 | |
| Lactobacillus crispatus | 100 | 1 Billion/dose (total |
| Lactobacillus rhamnosus | | lactobacilli claimed at |
| Lactobacillus gasseri | | the commercial expiry date) |
| Inulin | 25 | |
| Maltodextrin | 121 | |
| Magnesium stearate | 4 | |
| Silicon dioxide | 4 | |
| HPMC and titanium dioxide | 96 | |
| capsule | | |
| TOTAL | 550 | |

### Example 2d

| Ingredient | mg/dose | Probiotics Supply |
|---|---|---|
| Boric acid | 150 | |
| Lactobacillus crispatus | 100 | 1 Billion/dose (total |
| Lactobacillus rhamnosus | | lactobacilli claimed at |
| Lactobacillus gasseri | | the commercial expiry date) |
| Inulin | 25 | |
| Maltodextrin | 171 | |
| Magnesium stearate | 4 | |
| Silicon dioxide | 4 | |
| HPMC and titanium dioxide | 96 | |
| capsule | | |
| TOTAL | 550 | |

### Example 2e

| Ingredient | mg/dose | Probiotics Supply |
|---|---|---|
| Boric acid | 100 | |
| Lactobacillus crispatus | 100 | 1 Billion/dose (total |
| Lactobacillus rhamnosus | | lactobacilli claimed at |
| Lactobacillus gasseri | | the commercial expiry date) |
| Inulin | 25 | |
| Maltodextrin | 171 | |
| Magnesium stearate | 4 | |
| Silicon dioxide | 4 | |
| HPMC and titanium dioxide | 96 | |
| capsule | | |
| TOTAL | 500 | |

### Example 2f

| Ingredient | mg/dose | Probiotics Supply |
|---|---|---|
| Boric acid | 300 | |
| Lactobacillus crispatus | 100 | 1.5 Billion/dose (total |
| Lactobacillus rhamnosus | | lactobacilli claimed at |
| Lactobacillus gasseri | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 2g

| Ingredient | mg/dose | Probiotics Supply |
|---|---|---|
| Boric acid | 300 | |
| Lactobacillus crispatus | 100 | 2 Billion/dose (total |
| Lactobacillus rhamnosus | | lactobacilli claimed at |
| Lactobacillus gasseri | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 2h

| Ingredient | mg/dose | Probiotics Supply |
|---|---|---|
| Boric acid | 300 | |
| Lactobacillus crispatus | 100 | 2.5 Billion/dose (total |
| Lactobacillus rhamnosus | | lactobacilli claimed at |
| Lactobacillus gasseri | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

3. Formulas with 2 probiotic micro-organisms in association with boric acid (scalar concentrations of probiotics and boric acid):

### Example 3a

| Ingredient | mg/dose | Probiotics Supply |
|---|---|---|
| Boric acid | 300 | |
| Lactobacillus rhamnosus | 100 | 1 Billion/dose (total |
| Lactobacillus gasseri | | lactobacilli claimed at |
| | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 3b

| Ingredient | mg/dose | Probiotics Supply |
|---|---|---|
| Boric acid | 250 | |
| Lactobacillus rhamnosus | 100 | 1 Billion/dose (total |
| Lactobacillus gasseri | | lactobacilli claimed at |
| | | the commercial expiry date) |
| Inulin | 25 | |
| Maltodextrin | 71 | |
| Magnesium stearate | 4 | |
| Silicon dioxide | 4 | |
| HPMC and titanium dioxide | 96 | |
| capsule | | |
| TOTAL | 550 | |

### Example 3c

| Ingredient | mg/dose | Probiotics Supply |
|---|---|---|
| Boric acid | 200 | |
| Lactobacillus rhamnosus | 100 | 1 Billion/dose (total |
| Lactobacillus gasseri | | lactobacilli claimed at |
| | | the commercial expiry date) |
| Inulin | 25 | |
| Maltodextrin | 121 | |
| Magnesium stearate | 4 | |
| Silicon dioxide | 4 | |
| HPMC and titanium dioxide | 96 | |
| capsule | | |
| TOTAL | 550 | |

### Example 3d

| Ingredient | mg/dose | Probiotics Supply |
|---|---|---|
| Boric acid | 150 | |
| Lactobacillus rhamnosus | 100 | 1 Billion/dose (total |
| Lactobacillus gasseri | | lactobacilli claimed at |
| | | the commercial expiry date) |
| Inulin | 25 | |
| Maltodextrin | 171 | |
| Magnesium stearate | 4 | |
| Silicon dioxide | 4 | |
| HPMC and titanium dioxide | 96 | |
| capsule | | |
| TOTAL | 550 | |

### Example 3e

| Ingredient | mg/dose | Probiotics Supply |
|---|---|---|
| Boric acid | 300 | |
| Lactobacillus rhamnosus | 100 | 1.5 Billion/dose (total |
| Lactobacillus gasseri | | lactobacilli claimed at |
| | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 3f

| Ingredient | mg/dose | Probiotics Supply |
|---|---|---|
| Boric acid | 300 | |
| Lactobacillus rhamnosus | 100 | 2 Billion/dose (total |
| Lactobacillus gasseri | | lactobacilli claimed at |
| | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 3g

| Ingredient | mg/dose | Probiotics Supply |
|---|---|---|
| Boric acid | 300 | |
| Lactobacillus rhamnosus | 100 | 2.5 Billion/dose (total |
| Lactobacillus gasseri | | lactobacilli claimed at |
| | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 3h

| Ingredient | mg/dose | Probiotics Supply |
|---|---|---|
| Boric acid | 300 | |
| Lactobacillus rhamnosus | 100 | 0.5 Billion/dose (total |
| Lactobacillus gasseri | | lactobacilli claimed at |
| | | the commercial expiry date) |
| Inulin | 25 | |
| Maltodextrin | 21 | |
| Magnesium stearate | 4 | |
| Silicon dioxide | 4 | |
| HPMC and titanium dioxide | 96 | |
| capsule | | |
| TOTAL | 550 | |

### Example 4

### Effect of Boric Acid on Probiotic Micro-Organisms (Lactobacilli)

The possibility that the boric acid could be detrimental to the survival of the lactobacilli was assessed.

Single cultures, or in a mixture of four lactobacilli, were grown for 24 hours at 37° C. in an atmosphere with 5% CO₂, in MRS medium, with or without 0.625% of boric acid.

The results of the lactobacilli cultures counts, performed by flow cytometry (FACS), are shown in Table 1.

The obtained results demonstrate that boric acid has no inhibitory effect on the growth of the lactobacilli tested, either alone or in combination. These results demonstrate that the presence of boric acid in a composition comprising lactobacilli is not an obstacle to the survival of the same lactobacilli.

TABLE 1
Growth of Lactobacillus in the presence of 0.625% of boric acid

| Lactobacillus | MRS without boric acid | MRS with boric acid |
|---|---|---|
| crispatus | 1.19 × 10⁷ | 6.29 × 10⁶ |
| gasseri | 7.28 × 10⁶ | 2.37 × 10⁶ |
| jensenii | 7.63 × 10⁶ | 7.47 × 10⁶ |
| rhamnosus | 7.45 × 10⁶ | 3.17 × 10⁶ |
| mix | 7.63 × 10⁶ | 7.40 × 10⁶ |

### Example 5

### Synergistic Effect of the Mixture Comprising Boric Acid and Lactobacilli

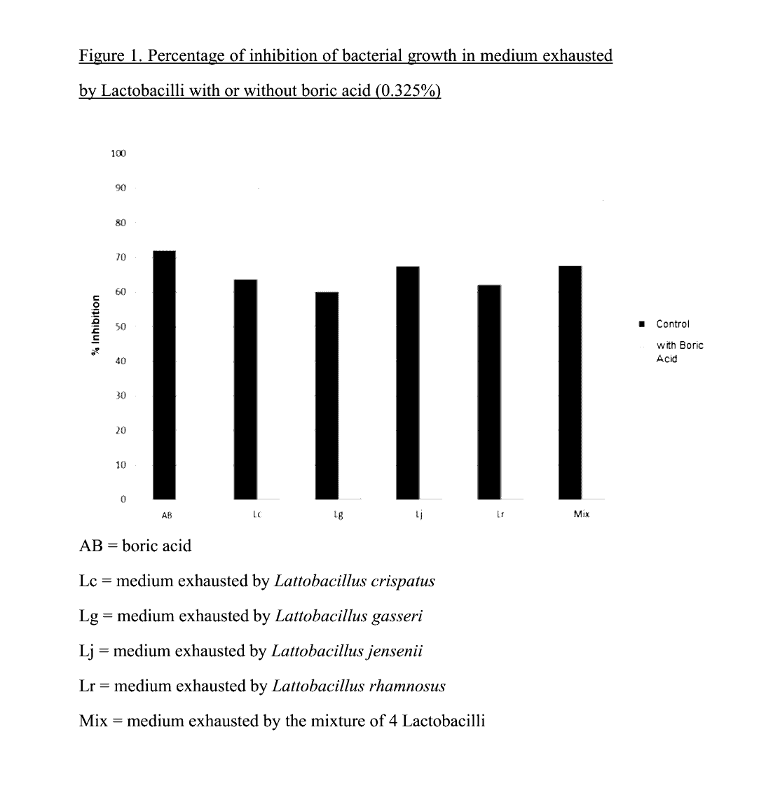

To evaluate the inhibitory effect of the mixture of one or more lactobacilli on the growth of Gardnerella vaginalis in the presence of boric acid, a strain of Gardnerella vaginalis was incubated for 24 hours in filtrate of exhausted Lactobacilli medium, in the presence or in the absence of boric acid (0.325%). The percentage growth inhibition results obtained from cultures of Gardnerella vaginalis are shown in FIG. 1.

The obtained results demonstrate that the inhibitory effect against the growth of Gardnerella vaginalis obtained in the presence of the combination of one or more lactobacilli with boric acid is greater than both boric acid alone and lactobacilli alone. These results demonstrate the synergistic effect provided by the composition comprising one or more lactobacilli in the presence of boric acid.

1. A composition comprising boric acid and a mixture of at least two lactobacilli selected from L. rhamnosus, L. crispatus, L. jensenii and L. gasseri.

2. The composition according to claim 1, characterized in that said mixture consists of two, three or four of said lactobacilli.

3. The composition according to claim 1, characterized in that said mixture consists of L. rhamnosus and L. gasseri.

4. The composition according to claim 1, characterized in that said mixture consists of L. rhamnosus, L. crispatus and L. gasseri.

5. The composition according to claim 1, characterized in that said mixture consists of L. rhamnosus, L. crispatus, L. jensenii and L. gasseri.

6. The composition according to claim 1, characterized in that boric acid is contained in said composition in an amount by weight comprised between 70% and 10%, preferably between 60% and 15%.

7. The composition according to claim 1, characterized in that the lactobacilli mixture is contained in said composition in an amount by weight comprised between 30% and 10%, preferably between 25% and 15%, about 20%.

8. The composition according to claim 3, characterized in that the total amount of lactobacilli L. rhamnosus and L. gasseri present in the mixture is comprised between 0.3 and 2.5 billion/dose, preferably between 0.5 and 2 billion/dose, more preferably between 1 and 1.5 billion/dose.

9. The composition according to claim 4, characterized in that the total amount of lactobacilli L. rhamnosus, L. crispatus and L. gasseri present in the mixture is comprised between 0.5 and 3.5 billion/dose, preferably between 0.8 and 3 billion/dose, more preferably between 1 and 2.5 billion/dose.

10. The composition according to claim 5, characterized in that the total amount of lactobacilli L. rhamnosus, L. crispatus, L. jensenii and L. gasseri present in the mixture is comprised between 0.5 and 3.5 billion/dose, preferably between 0.8 and 3 billion/dose, more preferably between 1 and 2.5 billion/dose.

11. The composition according to claim 1, further comprising at least one physiologically acceptable excipient.

12. The composition according to claim 11, characterized by being administrable by vaginal route.

13. The composition according to claim 12, characterized by being a capsule, an ovule, a cannula, a gel or a solution for intracavitary use, preferably a capsule or ovule.

14. A method for treating or preventing mycotic or bacterial vulvovaginitis, said method comprising administering an effective amount of a composition according to claim 1 to a subject in need thereof, thereby treating or preventing mycotic or bacterial vulvovaginitis in said subject.

15. A method for treating or preventing mycotic or bacterial vulvovaginitis, said method comprising administering an effective amount of a composition according to claim 11 to a subject in need thereof, thereby treating or preventing mycotic or bacterial vulvovaginitis in said subject.
